(19) United States
(12) Patent Application Publication
(10) Pub. No.: US 2025/0073487 A1
(43) Pub. Date: Mar. 6, 2025

(54) APPARATUS FOR LIGHT-BASED DIAGNOSTIC TREATMENT AND OPERATING METHOD THEREOF

(71) Applicants: KOREA INSTITUTE OF SCIENCE AND TECHNOLOGY, Seoul (KR); SEOUL NATIONAL UNIVERSITY R&DB FOUNDATION, Seoul (KR); SAMSUNG LIFE PUBLIC WELFARE FOUNDATION, Seoul (KR)

(72) Inventors: Sungwook YANG, Seoul (KR); Jin KIM, Seoul (KR); Myunghwan CHOI, Seongnam-si (KR); Kyuha CHONG, Seoul (KR)

(21) Appl. No.: 18/435,152

(22) Filed: Feb. 7, 2024

(30) Foreign Application Priority Data
Sep. 6, 2023 (KR) 10-2023-0118476

(51) Int. Cl. A61N 5/06; G06T 7/00

(52) U.S. Cl. CPC A61N 5/0616; G06T 7/0012; A61N 2005/0627; A61N 2005/0632; G06T 2207/30004

(57) ABSTRACT

A light-based diagnostic treatment apparatus and an operating method of a light-based diagnostic treatment apparatus are provided. The operating method of a light-based diagnostic treatment apparatus includes obtaining local images over time through a camera of the light-based diagnostic treatment apparatus, extracting an image feature point from the local images, generating a lesion map including a lesion area of a user by combining the local images based on the extracted image feature point, and generating a cumulative light irradiation map indicating accumulation information of therapeutic light output to the lesion area based on the lesion map and position information of an optical stimulation unit of the light-based diagnostic treatment apparatus.

### CROSS-REFERENCE TO RELATED APPLICATION

This application claims the benefit of Korean Patent Application No. 10-2023-0118476 filed on Sep. 6, 2023, in the Korean Intellectual Property Office, the entire disclosure of which is incorporated herein by reference for all purposes.

### BACKGROUND

### 1. Field of the Invention

The following disclosure relates to a light-based diagnostic treatment method.

### 2. Description of the Related Art

In conventional lesion treatment, such as a tumor, a lesion may be distinguished using a diagnostic apparatus. Thereafter, a person may perform an operation or procedure by manually recognizing a lesion position for treatment. It may be essential to match a coordinate system between a diagnostic image and current operation equipment to utilize radiation or a robot. However, an error may inevitably occur during a coordinate matching process. Specifically, for soft tissue, such as brain tissue, an image obtained before an operation may be different from an image during the operation due to a loss of cerebrospinal fluid, removal of a tumor, respiration, and the like. In addition, a conventional light diagnostic method using light may have low resolution, and since photodynamic therapy (PDT) depends on limited medication selectivity, a long time period of more than 10 minutes may be required. To compensate for such problems from the conventional methods, there is a demand for research.

### SUMMARY

According to an aspect, there is provided an operating method of a light-based diagnostic treatment apparatus, the operating method including obtaining local images over time through a camera of the light-based diagnostic treatment apparatus, extracting an image feature point from the local images, generating a lesion map including a lesion area of a user by combining the local images based on the extracted image feature point, and generating a cumulative light irradiation map indicating accumulation information of therapeutic light output to the lesion area based on the lesion map and position information of an optical stimulation unit of the light-based diagnostic treatment apparatus.

The generating of the cumulative light irradiation map includes estimating an amount of light irradiated to the lesion area by recording a moving path of a probe in the lesion area, wherein the light is therapeutic light output by the optical stimulation unit, and generating the cumulative light irradiation map by displaying the estimated amount of light on the lesion map.

The generating of the lesion map includes generating the lesion map by connecting the local images based on a corresponding image feature point extracted from the local images.

The operating method further includes recognizing a shape of the lesion area based on the lesion map, determining an output pattern of therapeutic light based on the recognized lesion area, and outputting patterned therapeutic light based on the determined output pattern, wherein the shape of the lesion area corresponds to an output pattern of the patterned therapeutic light.

The outputting of the patterned therapeutic light includes determining an intensity of the patterned therapeutic light to be output to each predetermined position in the lesion area based on an accumulated amount of the patterned therapeutic light at each predetermined position in the lesion area displayed on a cumulative light irradiation map, and outputting therapeutic light having the determined intensity of therapeutic light through the optical stimulation unit.

According to an aspect, there is provided an operating method of a light-based diagnostic treatment apparatus including a lesion detector and a therapeutic light generator sharing a single optical path, the operating method including obtaining a first lesion image of a lesion area of a user based on an optical signal transmitted through the single optical path, detecting the lesion area from the obtained first lesion image, in response to termination of detecting the lesion area, transmitting a first trigger signal to the therapeutic light generator, in response to reception of the first trigger signal by the therapeutic light generator, generating patterned therapeutic light based on the shape of the lesion area, and outputting the patterned therapeutic light to the lesion area through the single optical path.

The operating method further includes, in response to termination of outputting the patterned therapeutic light, transmitting a second trigger signal to a lesion image obtainer, in response to reception of the second trigger signal by the lesion detector, obtaining a second lesion image of the lesion area based on an optical signal transmitted through the single optical path, and detecting the lesion area from the second lesion image.

The generating of the patterned therapeutic light includes determining an output pattern of therapeutic light based on the shape of the lesion area, and outputting patterned therapeutic light based on the determined output pattern, wherein the shape of the lesion area corresponds to an output pattern of the patterned therapeutic light.

According to an aspect, there is provided a light-based diagnostic treatment apparatus including a lesion image obtainer configured to obtain local images over time through a camera of the light-based diagnostic treatment apparatus, an image feature point extractor configured to extract an image feature point from the local images, a lesion map generator configured to generate a lesion map including a lesion area of a user by combining the local images based on the extracted image feature point, and a cumulative light irradiation map configured to generate a cumulative light irradiation map indicating accumulation information of therapeutic light output to the lesion area based on the lesion map and position information of an optical stimulation unit of the light-based diagnostic treatment apparatus over time.

The cumulative light irradiation map generator is configured to estimate an amount of light irradiated to the lesion area by recording a moving path of a probe in the lesion area, wherein the light is therapeutic light output by the optical stimulation unit, and generate the cumulative light irradiation map by displaying the estimated amount of light on the lesion map.

The lesion map generator is configured to generate the lesion map by connecting the local images based on a corresponding image feature point extracted from the local images.

The light-based diagnostic treatment apparatus further includes a therapeutic light generator configured to recognize a shape of the lesion area based on the lesion map, determine an output pattern of therapeutic light based on the recognized shape of the lesion area, and output patterned therapeutic light based on the determined output pattern, wherein the shape of the lesion area corresponds to an output pattern of the patterned therapeutic light.

The therapeutic light generator is configured to determine an intensity of the patterned therapeutic light to be output to each predetermined position in the lesion area based on an accumulated amount of the patterned therapeutic light at each predetermined position in the lesion area displayed on a cumulative light irradiation map, and output therapeutic light having the determined intensity of therapeutic light through the optical stimulation unit.

According to an aspect, there is provided a light-based diagnostic treatment apparatus including a lesion detector configured to obtain a first lesion image of a lesion area of a user based on an optical signal transmitted through a single optical path, detect the lesion area from the obtained first lesion image, and in response to termination of detecting the lesion area, transmit a first trigger signal to a therapeutic light generator, and the therapeutic light generator configured to in response to reception of the first trigger signal, generate patterned therapeutic light based on a shape of the lesion area, and output the patterned therapeutic light to the lesion area through the single optical path.

The therapeutic light generator is configured to, in response to termination of outputting the patterned therapeutic light, transmit a second trigger signal to the lesion detector, and the lesion detector is configured to, in response to reception of the second trigger signal, obtain a second lesion image of the lesion area based on an optical signal transmitted through the single optical path and detect the lesion area from the obtained second lesion image.

The therapeutic light generator is configured to determine an output pattern of therapeutic light based on a shape of the lesion area, and generate patterned therapeutic light based on the determined output pattern, wherein the shape of the lesion area corresponds to an output pattern of the patterned therapeutic light.

According to one embodiment, both a diagnosis function and a treatment function for a lesion may be provided through a single apparatus.

According to one embodiment, a system control framework related to a photodynamic therapy apparatus, which is able to analyze a light diagnostic image in real-time and irradiate a patterned beam, may be established.

According to one embodiment, since a single apparatus is able to provide light diagnosis and photodynamic therapy, an error that may occur during a process of matching coordinates between a diagnostic apparatus and a treatment apparatus in a conventional method may be removed.

According to one embodiment, a cumulative light irradiation map and a lesion map indicating a lesion may be generated based on a local image obtained through a probe.

According to one embodiment, a lesion map may be generated by scanning an entirety of a lesion area and whether to treat or an amount of light to be transmitted may be determined by recording a current position of a probe.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 is a diagram illustrating an overview of an apparatus for light-based diagnostic treatment according to one embodiment;

FIG. 2 is a flowchart illustrating an operating method of an apparatus for light-based diagnostic treatment according to one embodiment;

FIG. 3 is a flowchart illustrating an operating method of an apparatus for light-based diagnostic treatment according to one embodiment;

FIG. 4 is a diagram illustrating patterned light according to one embodiment;

FIGS. 5A and 5B are diagrams illustrating a process of generating a lesion map and a cumulative light irradiation map according to one embodiment; and

FIG. 6 is a diagram illustrating a configuration of an apparatus for light-based diagnostic treatment according to one embodiment.

### DETAILED DESCRIPTION

The following detailed structural or functional description is provided as an example only and various alterations and modifications may be made to the embodiments. Here, the embodiments are not construed as limited to the disclosure and should be understood to include all changes, equivalents, and replacements within the idea and the technical scope of the disclosure.

Terms, such as first, second, and the like, may be used herein to describe components. Each of these terminologies is not used to define an essence, order or sequence of a corresponding component but used merely to distinguish the corresponding component from other component(s). For example, a first component may be referred to as a second component, and similarly the second component may also be referred to as the first component.

It should be noted that if it is described that one component is “connected”, “coupled”, or “joined” to another component, a third component may be “connected”, “coupled”, and “joined” between the first and second components, although the first component may be directly connected, coupled, or joined to the second component.

The singular forms “a,” “an,” and “the” are intended to include the plural forms as well, unless the context clearly indicates otherwise. As used herein, “A or B”, “at least one of A and B”, “at least one of A or B”, “A, B or C”, “at least one of A, B and C”, and “at least one of A, B, or C,” each of which may include any one of the items listed together in the corresponding one of the phrases, or all possible combinations thereof. It will be further understood that the terms “comprises/including” and/or “includes/including” when used herein, specify the presence of stated features, integers, operations, operations, elements, and/or components, but do not preclude the presence or addition of one or more other features, integers, operations, operations, elements, components and/or groups thereof.

Unless otherwise defined, all terms, including technical and scientific terms, used herein have the same meaning as commonly understood by one of ordinary skill in the art to which this disclosure pertains. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

As used in connection with the present disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an example, the module may be implemented in a form of an application-specific integrated circuit (ASIC).

The term “unit” used herein may refer to a software or hardware component, such as a field-programmable gate array (FPGA) or an ASIC, and the “unit” performs predefined functions. However, “unit” is not limited to software or hardware. The “unit” may be configured to reside on an addressable storage medium or configured to operate one or more processors. Accordingly, the “unit” may include, for example, components, such as software components, object-oriented software components, class components, and task components, processes, functions, attributes, procedures, sub-routines, segments of program code, drivers, firmware, microcode, circuitry, data, databases, data structures, tables, arrays, and variables. The functionalities provided in the components and “units” may be combined into fewer components and “units” or may be further separated into additional components and “units.” Furthermore, the components and “units” may be implemented to operate on one or more central processing units (CPUs) within a device or a security multimedia card. In addition, “unit” may include one or more processors.

Hereinafter, the examples will be described in detail with reference to the accompanying drawings. When describing the embodiments with reference to the accompanying drawings, like reference numerals refer to like elements and a repeated description related thereto will be omitted.

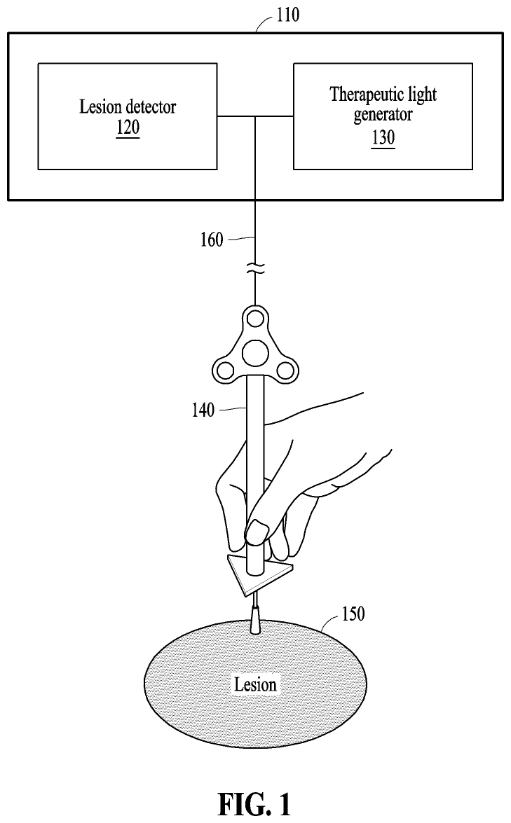

FIG. 1 is a diagram illustrating an overview of an apparatus for light-based diagnostic treatment according to one embodiment.

An apparatus for light-based diagnostic treatment (hereinafter, also referred to as a light-based diagnostic treatment apparatus) described herein may observe tissue in real time, may analyze a lesion, and may treat by selectively irradiating light, that is, therapeutic light. In addition, when freely scanning over tissue such as a handheld scanner, the apparatus for light-based diagnostic treatment may diagnose a lesion in real time using an imaging function and may irradiate local therapeutic light to a diagnosed lesion area by activating a treatment function in a short time. The function of scanning and diagnosing a lesion and the treatment function may be performed by a probe sharing the same optical path. The apparatus for light-based diagnostic treatment may simultaneously perform image acquisition and treatment and may perform through a single apparatus and a single optical path. The apparatus for the light-based diagnostic treatment described herein may perform a real-time control method of interleaving light for obtaining a lesion image and therapeutic light using a single optical path in common. In addition, the apparatus for the light-based diagnostic treatment may generate an expanded lesion map for an entire lesion area based on a local image having a narrow field of view (FOV) for a precise lesion diagnosis, which may determine whether to treat for each area by recording a position of a current position of a diagnostic probe and may determine an amount of transmitted light to each area. The apparatus for light-based diagnostic treatment may generate a lesion map by obtaining a local image including a lesion area or a lesion image based on an optical signal transmitted through a single optical path and may generate a cumulative light irradiation map indicating information of accumulation of the therapeutic light output to the lesion area based on the lesion map and position information of a photic stimulation unit of the apparatus for light-based diagnostic treatment over time. In addition, the apparatus for light-based diagnostic treatment may interleave an operation of generating a lesion map by obtaining a local image or a lesion image, an operation of detecting a lesion area, and an operation of outputting therapeutic light to a lesion area, based on a trigger signal. Two operations may be interleaved in a significantly short time. The lesion described herein may refer to a brain lesion, but is not limited thereto.

Referring to FIG. 1, a light-based diagnostic treatment apparatus 110 in one embodiment may include a lesion detector 120 and a therapeutic light generator 130. The lesion detector 120 and the therapeutic light generator 130 may be connected to a probe 140. An optical signal of the lesion detector 120 and therapeutic light of the therapeutic light generator 130 may be transmitted through a single optical path 160, and the single optical path 160 may be connected to the probe 140 as an optical fiber bundle or may be included in the probe 140.

The lesion detector 120 may obtain a first lesion image of a lesion area 150 of a user based on the optical signal transmitted through the single optical path 160. The lesion area 150 may be detected from the obtained first lesion image and in response to termination of detection of the lesion area 150, a first trigger signal may be transmitted to the therapeutic light generator 130.

In response to reception of the first trigger signal, the therapeutic light generator 130 may generate patterned therapeutic light based on a shape of the lesion area 150 and may output the patterned therapeutic light to the lesion area 150 through the single optical path 160. The therapeutic light generator 130 may determine an output pattern of therapeutic light based on the shape of the lesion area 150 and may generate patterned therapeutic light based on the determined output pattern.

In response to termination of outputting the patterned therapeutic light, the therapeutic light generator 130 may transmit a second trigger signal to the lesion detector 120. In response to reception of the second trigger signal, the lesion detector 120 may obtain a second lesion image of the lesion area 150 based on an optical signal transmitted through the single optical path 160 and may detect the lesion area 150 from the obtained second lesion image. An operation of the therapeutic light generator 130 and an operation of the lesion detector 120 performed through the single optical path 160 may be interleaved through a trigger signal including the first trigger signal and the second trigger signal.

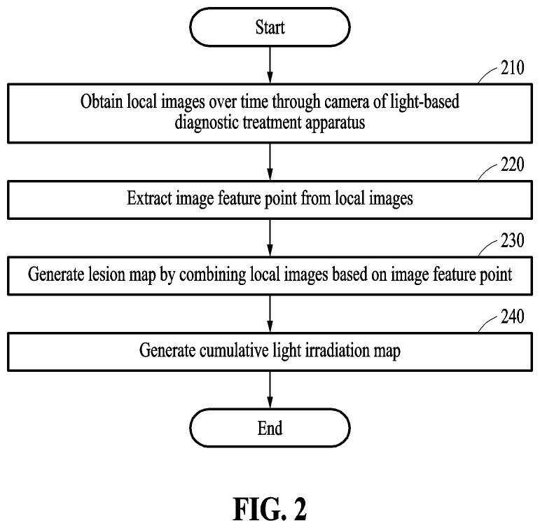

FIG. 2 is a flowchart illustrating an operating method of an apparatus for light-based diagnostic treatment according to one embodiment.

Referring to FIG. 2, in one embodiment, in operation 210, a light-based diagnostic treatment apparatus may obtain local images over time through a camera of the light-based diagnostic treatment apparatus. The light-based diagnostic treatment apparatus may obtain a local image, for example, less than or equal to 5 mm, using a camera. That is, the light-based diagnostic treatment apparatus may obtain local images over time by moving over time in a lesion area of a user (or a patient). Since the light-based diagnostic treatment apparatus collects local images while moving on the lesion area over time, local images of the lesion area that gradually expands over time may be collected. Since the local image is an image generated with respect to the lesion area, the local image may be referred to as a lesion image.

In operation 220, the light-based diagnostic treatment apparatus may extract an image feature point from the local images by performing pre-processing on the local images. For example, the light-based diagnostic treatment apparatus may extract an image feature point from the local images using a scale invariant feature transform (SIFT) method. The light-based diagnostic treatment apparatus may extract an image feature point from the local images as well as perform pre-processing and other related operations.

In operation 230, the light-based diagnostic treatment apparatus may generate a lesion map including a lesion area of a user by combining the local images based on the extracted image feature point. More specifically, the light-based diagnostic treatment apparatus may generate a lesion map by connecting the local images based on corresponding image feature points among extracted image feature points. That is, the light-based diagnostic treatment apparatus may generate a lesion map by connecting local images including at least one identical image feature point based on the identical image feature point. An operation of the light-based diagnostic treatment apparatus to combine local images based on an image feature point may be referred to as mapping, stitching, overlapping, or connecting. However, the example is not limited to the description provided herein. The light-based diagnostic treatment apparatus may generate a lesion map by combining local images obtained over time based on the image feature point. Since the light-based diagnostic treatment apparatus collects the local images by moving over the lesion area over time, the light-based diagnostic treatment apparatus may generate a lesion map for a wider lesion area as time elapses.

In operation 240, the light-based diagnostic treatment apparatus may generate a cumulative light irradiation map indicating accumulation information on therapeutic light output to the lesion area based on the lesion map and position information of an optical stimulation unit of the light-based diagnostic treatment apparatus over time.

The light-based diagnostic treatment apparatus may estimate an amount of light irradiated to the lesion area by recording a moving path of a probe in the lesion area over time, wherein the light is therapeutic light output by the optical stimulation unit. The light-based diagnostic treatment apparatus may generate the cumulative light irradiation map by displaying an estimated amount of light on the lesion map. That is, the cumulative light irradiation map may display at least one of whether therapeutic light is irradiated to each predetermined position of the lesion area and accumulation information on irradiated therapeutic light at each predetermined position in the lesion area.

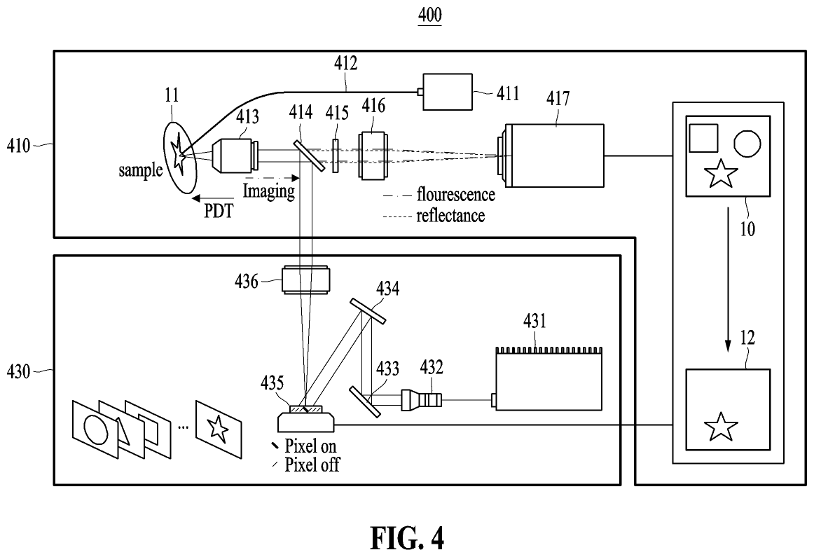

The light-based diagnostic treatment apparatus may recognize a shape of the lesion area based on the lesion map. More specifically, the light-based diagnostic treatment apparatus may recognize at least one of a shape and a state of the lesion area based on the lesion map. The light-based diagnostic treatment apparatus may determine an output pattern of the therapeutic light based on the shape of the recognized lesion area and may output patterned therapeutic light based on the determined output pattern. The shape of the lesion area may correspond to an output pattern of the patterned therapeutic light. In addition, at least one of the shape and the state of the lesion area may correspond to the output pattern of the patterned therapeutic light, and the output pattern of the patterned therapeutic light may be determined based on at least one of the shape and the state of the lesion area. The light-based diagnostic treatment apparatus may detect the shape of the lesion area by performing binauralization on the lesion map and may convert coordinates used to detect the lesion area or the shape of the lesion area into coordinates to output therapeutic light. The light-based diagnostic treatment apparatus may output patterned therapeutic light for the shape of the detected lesion area based on the converted coordinates. The lesion area to which the therapeutic light is irradiated may glow. A detailed description related to the patterned therapeutic light based on the output pattern may be provided with reference to FIG. 4.

In the embodiment of FIG. 2, the light-based diagnostic treatment apparatus may determine the intensity of the patterned therapeutic light to be output to each predetermined position in the lesion area based on an accumulated amount of the patterned therapeutic light at each predetermined position in the lesion area displayed on the cumulative light irradiation map. The light-based diagnostic treatment apparatus may output the therapeutic light having a determined intensity of therapeutic light through the optical stimulation unit. For example, the light-based diagnostic treatment apparatus may determine the intensity of the patterned therapeutic light to be output to each predetermined position to prevent from exceeding or falling short of a threshold value of accumulation information based on the accumulation information on the therapeutic light of each predetermined position. The light-based diagnostic treatment apparatus may determine the intensity of the patterned therapeutic light to be output to each position to prevent the therapeutic light from being irradiated too much or too little to each position in the lesion area. The light-based diagnostic treatment apparatus may determine the intensity of the patterned therapeutic light to be output to each position differently.

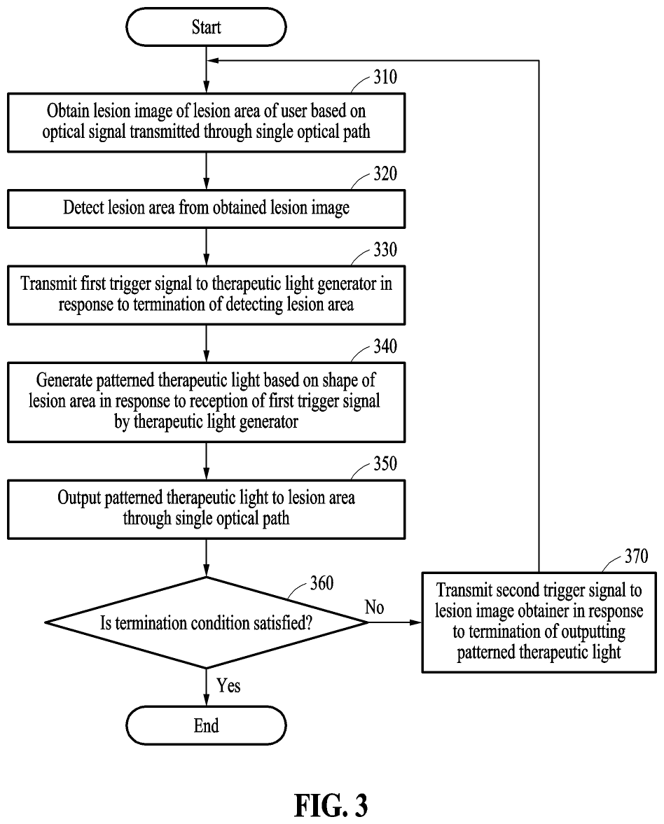

FIG. 3 is a flowchart illustrating an operating method of an apparatus for light-based diagnostic treatment according to one embodiment.

A light-based diagnostic treatment apparatus may include a lesion detector and a therapeutic light generator sharing a single optical path. Referring to FIG. 3, in operation 310, the light-based diagnostic treatment apparatus may obtain a first lesion image of a lesion area of a user based on an optical signal transmitted through the single optical path. In operation 320, the light-based diagnostic treatment apparatus may detect a lesion area (or a region of interest (ROI)) from the obtained first lesion image.

In operation 330, in response to termination of detecting the lesion area, the light-based diagnostic treatment apparatus may transmit a first trigger signal to the therapeutic light generator. In operation 340, in response to reception of the first trigger signal by the therapeutic light generator, the light-based diagnostic treatment apparatus may generate patterned therapeutic light based on the shape of the lesion area. The light-based diagnostic treatment apparatus may determine an output pattern of therapeutic light based on the shape of the lesion area. The shape of the lesion area may correspond to an output pattern of the patterned therapeutic light. The process of generating the patterned therapeutic light is described with reference to FIG. 4.

Referring to FIG. 3, in operation 350, the light-based diagnostic treatment apparatus may output the patterned therapeutic light to the lesion area through the single optical path. The light-based diagnostic treatment apparatus may output the patterned therapeutic light based on the determined output pattern.

In operation 360, the light-based diagnostic treatment apparatus may identify whether a termination condition is satisfied. In this case, the termination condition may include a condition related to, for example, completion of treatment.

When it is determined that the termination condition is satisfied, in operation 370, the light-based diagnostic treatment apparatus may transmit a second trigger signal to a lesion image obtainer in response to termination of outputting the patterned therapeutic light.

In response to reception of the second trigger signal by the lesion detector, the light-based diagnostic treatment apparatus may perform operation 310 again. The light-based diagnostic treatment apparatus may obtain a second lesion image of the lesion area based on the optical signal transmitted through the single optical path. In addition, the light-based diagnostic treatment apparatus may detect the lesion area from the second lesion image. The light-based diagnostic treatment apparatus may repeat operations 310 to 370 of FIG. 3 until the termination condition is satisfied.

When it is determined that the termination condition is satisfied in operation 360, the light-based diagnostic treatment apparatus may terminate the process described above. In this embodiment, when the lesion image including the first lesion image and the second lesion image is locally collected, the lesion image may be referred to as a local image.

The operating method of the light-based diagnostic treatment apparatus according to another embodiment described in FIG. 2 and the operating method of the light-based diagnostic treatment apparatus according to one embodiment described in FIG. 3 may be performed by the same light-based diagnostic treatment apparatus, or after an operation of generating a lesion map of the operating method of the light-based diagnostic treatment apparatus according to another embodiment is performed as a pre-condition, an operation of the light-based diagnostic treatment apparatus according to another embodiment except for the operation of generating a lesion map and an operation of the light-based diagnostic treatment apparatus according to one embodiment may be performed as a series of operations.

FIG. 4 is a diagram illustrating patterned light according to one embodiment.

Referring to FIG. 4, a light-based diagnostic treatment apparatus 400 according to one embodiment may include a lesion detector 410 and a therapeutic light generator 430.

The lesion detector 410 may include a light source 411 and optical fiber (or an optical fiber bundle) 412 connected to the light source 411 and transmitting light to a lesion area. In addition, the lesion detector 410 may include one or more lenses 413 and 416, a mirror 414, a filter 415, and an image sensor 417. Reflected light, scattered light, or fluorescence from the lesion area may be collected through the first lens 413, and the collected reflected light, scattered light, or fluorescence may be transmitted to the image sensor 417 through the mirror 414, the filter 415, and the second lens 416, and may be used to generate a lesion image 10.

The lesion detector 410 may analyze the lesion image 10 generated by the image sensor 417 and may extract and diagnose a lesion area 11.

The therapeutic light generator 430 may include therapeutic light source 431 outputting therapeutic light, a beam expander 432 expanding a diameter of output therapeutic light, one or more mirrors 433 and 434, a patterning unit 435 forming patterned therapeutic light corresponding to the lesion area 11 by patterning the therapeutic light, and a third lens 436.

A diameter of the therapeutic light output by the therapeutic light source 431 may expand through the beam expander 432, and the expanded therapeutic light may be transmitted to the patterning unit 431 through the one or more mirrors 433 and 434. The patterning unit 431 may form patterned therapeutic light corresponding to the lesion area 11 based on a pattern or a shape 12 of the lesion area 11 and may irradiate the patterned therapeutic light to the lesion area 11 through the third lens 436, the mirror 414, and the first lens 413.

According to one embodiment, the first lens 413 may be an objective lens, the second lens 416, and the third lens 436 may be tube lenses.

In addition, according to one embodiment, when the image sensor 417 includes a point detector, a scanner may be included between the first lens 413 and the second lens 416.

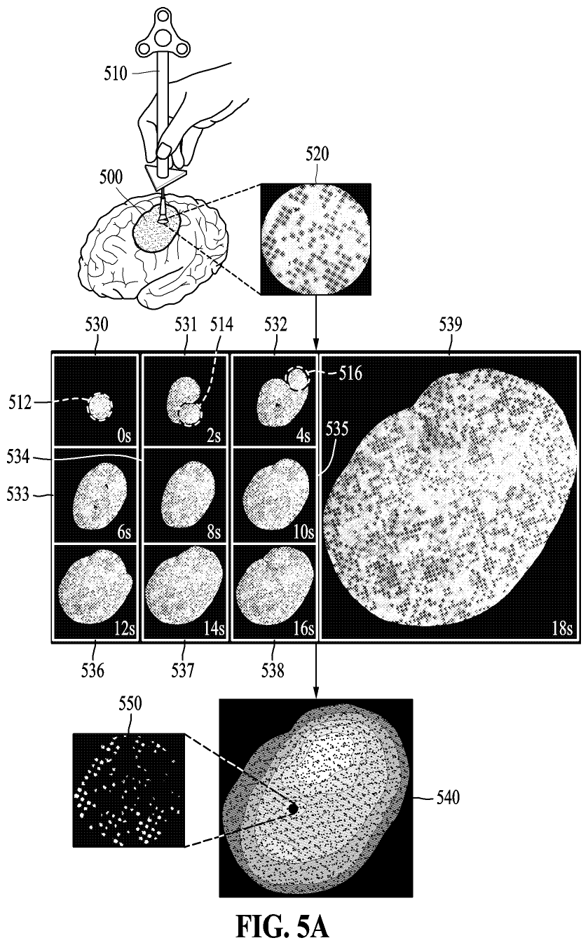

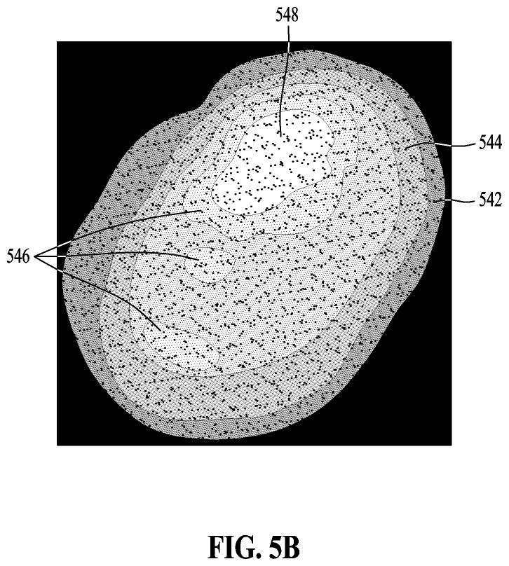

FIGS. 5A and 5B are diagrams illustrating a process of generating a lesion map and a cumulative light irradiation map according to one embodiment.

Referring to FIG. 5A, a probe 510 may be connected to a light-based diagnostic treatment apparatus. The medical staff may hold the probe 510 in their hand and may obtain local images from a lesion area 500, may generate a lesion map and a cumulative light irradiation map, and may irradiate therapeutic light to the lesion area 500. The probe 510 may include a soft fiber bundle. The light-based diagnostic treatment apparatus may continue to collect local images having a narrow FOV over time and may generate a lesion map including an entirety of the lesion area by combining the local images.

The light-based diagnostic treatment apparatus may obtain a local image having a narrow FOV. The local image may be, for example, the same as the reference number 520. The size of the local image obtained at one time may be, for example, about 5 mm. However, the example is not limited to the description provided herein.

The light-based diagnostic treatment apparatus may collect the local images over time through a camera connected to the probe. The light-based diagnostic treatment apparatus may extract an image feature point from the collected local images over time and may generate a lesion map by combining the local images based on the image feature point. The local image may be an image captured at one time while the probe stops over the lesion area. The probe may collect a plurality of local images including reference numbers 512, 514, and 516 while moving over the lesion area of the user over time and may generate a lesion map by combining the collected local images based on the image feature point. The lesion map may be referred to as a global map or a wide area map.

Reference numbers 530, 531, 532, 533, 534, 535, 536, 537, 538, and 539 may be lesion maps generated at a predetermined time interval. In the embodiment of FIG. 5A, the predetermined time interval may be 2 seconds but is not limited thereto. The light-based diagnostic treatment apparatus may generate the lesion map 531 by combining local images obtained from the lesion area for two seconds, based on the image feature point. In addition, the light-based diagnostic treatment apparatus may generate the lesion map 532 by combining local images obtained from the lesion area for four seconds based on the image feature point and may generate the lesion map 533 by combining local images obtained from the lesion area for six seconds based on the image feature point. The light-based diagnostic treatment apparatus may obtain local images over time and may generate the lesion map 539 by repeating a process of combining the local images based on an image feature point extracted from the obtained local images for 18 seconds. When the lesion area is wider and the light-based diagnostic treatment apparatus collects local images for longer than 18 seconds, the light-based diagnostic treatment apparatus may generate a larger lesion map than the lesion maps shown in FIG. 5A.

The light-based diagnostic treatment apparatus may generate a cumulative light irradiation map 540 indicating accumulation information on therapeutic light output to the lesion area based on the lesion map and position information of an optical stimulation unit of the light-based diagnostic treatment apparatus over time. The cumulative light irradiation map 540 may indicate accumulation information of therapeutic light output to the lesion area as a binary pattern 550, or by color or grouping.

The cumulative light irradiation map 540 expressed in a group is shown in FIG. 5B. Referring to FIG. 5B, areas having the same accumulation information of therapeutic light may be grouped as the same area or may be displayed in the same color. That is, areas included in an area 542 may have the same accumulation of information of therapeutic light. In addition, areas included in an area 544 may have the same accumulation information of therapeutic light, and areas included in an area 546 may have the same accumulation information of therapeutic light In addition, areas included in an area 548 may have the same accumulation information of therapeutic light. In this case, the same accumulation information of therapeutic light may refer to that the same amount of therapeutic light is irradiated.

An operation of generating a lesion map and an operation of generating a cumulative light irradiation map may be simultaneously or sequentially performed.

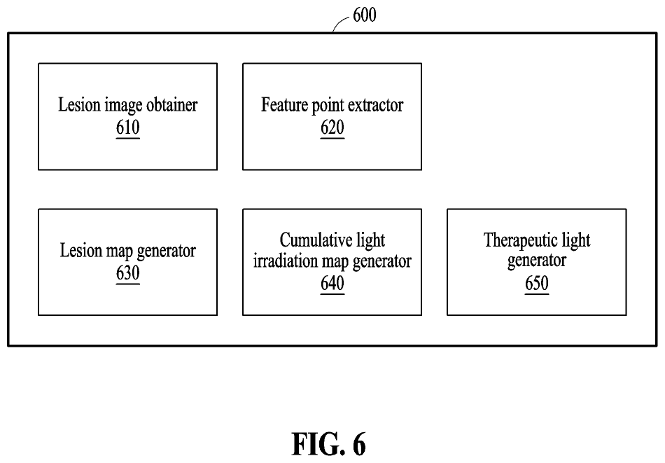

FIG. 6 is a diagram illustrating a configuration of an apparatus for light-based diagnostic treatment according to one embodiment.

Referring to FIG. 6, a light-based diagnostic treatment apparatus 600 may include a lesion image obtainer 610, a feature point extractor 620, a lesion map generator 630, a cumulative light irradiation map generator 640, and a therapeutic light generator 650. In this case, the light-based diagnostic treatment apparatus 600 may correspond to the light-based diagnostic treatment apparatus described in the present disclosure. In one embodiment, the light-based diagnostic treatment apparatus 600 may include a processor, a memory, and operations of the feature point extractor 620, the lesion map generator 630, and the cumulative light irradiation map generator 640 may be performed by the processor.

The lesion image obtainer 610 may obtain local images over time through a camera of the light-based diagnostic treatment apparatus. The lesion image obtainer 610 may include a camera or may be connected to the camera. The feature point extractor 620 may extract an image feature point from the local images. The lesion map generator 630 may generate a lesion map including a lesion area of a user by combining the local images based on the extracted image feature point. The lesion image obtainer 610 may generate a lesion map by connecting the local images based on corresponding image feature points among extracted image feature points.

The cumulative light irradiation map generator 640 may generate a cumulative light irradiation map indicating accumulation information on therapeutic light output to the lesion area based on the lesion map and position information of an optical stimulation unit of the light-based diagnostic treatment apparatus over time. The cumulative light irradiation map generator 640 may estimate an amount of light, which is therapeutic light output by an optical stimulation unit, irradiated to a lesion area by recording a moving path of a probe in the lesion area over time, and may generate a cumulative light irradiation map by displaying the estimated amount of light on a lesion map.

The therapeutic light generator 650 may recognize a shape of the lesion area based on the lesion map, may determine an output pattern of the therapeutic light based on the recognized shape of the lesion area, and may output patterned therapeutic light based on the determined output pattern. The therapeutic light generator 650 may determine the intensity of the patterned therapeutic light to be output to each predetermined position in the lesion area based on an accumulated amount of patterned therapeutic light at each predetermined position in the lesion area displayed on the cumulative light irradiation map and may output therapeutic light having the determined intensity of the therapeutic light through the optical stimulation unit.

The units described herein may be implemented using a hardware component, a software component and/or a combination thereof. A processing device may be implemented using one or more general-purpose or special-purpose computers, such as, for example, a processor, a controller and an arithmetic logic unit (ALU), a DSP, a microcomputer, an FPGA, a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or one or more combinations thereof, to independently or collectively instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described devices may be configured to act as one or more software modules in order to perform the operations of the above-described examples, or vice versa.

A number of example embodiments have been described above. Nevertheless, it should be understood that various modifications may be made to these example embodiments. For example, suitable results may be achieved if the described techniques are performed in a different order and/or if components in a described system, architecture, device, or circuit are combined in a different manner and/or replaced or supplemented by other components or their equivalents.

Accordingly, other implementations are within the scope of the following claims.

### STATEMENT REGARDING PRIOR DISCLOSURES BY THE INVENTOR OR A JOINT INVENTOR

The inventors of the present application have made related disclosure in Jin Kim et al., “Real-Time Control Framework of High-Speed Photo-Theranosis System,” KSME22, Nov. 11, 2022 and Jin Kim et al., “Real-Time Endomicroscopic Image Mosaicking with an EKF-based Sensor Fusion Approach,” EMBC 223 July 2023. The related disclosure was made less than one year before the effective filing date (Sep. 6, 2023) of the present application and the inventors of the present application are the same as those of the related disclosure. Accordingly, the related disclosure is disqualified as prior art under 35 USC 102(a)(1) against the present application. See 35 USC 102(b)(1)(A).

What is claimed is:

1. An operating method of a light-based diagnostic treatment apparatus, the operating method comprising:
obtaining local images over time through a camera of the light-based diagnostic treatment apparatus;
extracting an image feature point from the local images;
generating a lesion map comprising a lesion area of a user by combining the local images based on the extracted image feature point; and
generating a cumulative light irradiation map indicating accumulation information of therapeutic light output to the lesion area based on the lesion map and position information of an optical stimulation unit of the light-based diagnostic treatment apparatus.

2. The operating method of claim 1, wherein the generating of the cumulative light irradiation map comprises:
estimating an amount of light irradiated to the lesion area by recording a moving path of a probe in the lesion area, wherein the light is therapeutic light output by the optical stimulation unit; and
generating the cumulative light irradiation map by displaying the estimated amount of light on the lesion map.

3. The operating method of claim 1, wherein the generating of the lesion map comprises:
generating the lesion map by connecting the local images based on a corresponding image feature point extracted from the local images.

4. The operating method of claim 1, further comprising:
recognizing a shape of the lesion area based on the lesion map;
determining an output pattern of therapeutic light based on the recognized lesion area; and
outputting patterned therapeutic light based on the determined output pattern,
wherein the shape of the lesion area corresponds to an output pattern of the patterned therapeutic light.

5. The operating method of claim 4, wherein the outputting of the patterned therapeutic light comprises:
determining an intensity of the patterned therapeutic light to be output to each predetermined position in the lesion area based on an accumulated amount of the patterned therapeutic light at each predetermined position in the lesion area displayed on a cumulative light irradiation map; and
outputting therapeutic light having the determined intensity of therapeutic light through the optical stimulation unit.

6. An operating method of a light-based diagnostic treatment apparatus comprising a lesion detector and a therapeutic light generator sharing a single optical path, the operating method comprising:
obtaining a first lesion image of a lesion area of a user based on an optical signal transmitted through the single optical path;
detecting the lesion area from the obtained first lesion image;
in response to termination of detecting the lesion area, transmitting a first trigger signal to the therapeutic light generator;
in response to reception of the first trigger signal by the therapeutic light generator, generating patterned therapeutic light based on the shape of the lesion area; and
outputting the patterned therapeutic light to the lesion area through the single optical path.

7. The operating method of claim 6, further comprising:
in response to termination of outputting the patterned therapeutic light, transmitting a second trigger signal to a lesion image obtainer;
in response to reception of the second trigger signal by the lesion detector, obtaining a second lesion image of the lesion area based on an optical signal transmitted through the single optical path; and
detecting the lesion area from the second lesion image.

8. The operating method of claim 6, wherein the generating of the patterned therapeutic light comprises:
determining an output pattern of therapeutic light based on the shape of the lesion area; and
outputting patterned therapeutic light based on the determined output pattern,
wherein the shape of the lesion area corresponds to an output pattern of the patterned therapeutic light.

9. A non-transitory computer-readable storage medium storing instructions that, when executed by a processor, cause the processor to perform the operating method of claim 1.

10. A light-based diagnostic treatment apparatus, comprising:
a lesion image obtainer configured to obtain local images over time through a camera of the light-based diagnostic treatment apparatus;
an image feature point extractor configured to extract an image feature point from the local images;
a lesion map generator configured to generate a lesion map comprising a lesion area of a user by combining the local images based on the extracted image feature point; and
a cumulative light irradiation map configured to generate a cumulative light irradiation map indicating accumulation information of therapeutic light output to the lesion area based on the lesion map and position information of an optical stimulation unit of the light-based diagnostic treatment apparatus over time.

11. The light-based diagnostic treatment apparatus of claim 10, wherein the cumulative light irradiation map generator is configured to:
estimate an amount of light irradiated to the lesion area by recording a moving path of a probe in the lesion area, wherein the light is therapeutic light output by the optical stimulation unit, and
generate the cumulative light irradiation map by displaying the estimated amount of light on the lesion map.

12. The light-based diagnostic treatment apparatus of claim 10, wherein the lesion map generator is configured to:
generate the lesion map by connecting the local images based on a corresponding image feature point extracted from the local images.

13. The light-based diagnostic treatment apparatus of claim 10, further comprising:
a therapeutic light generator configured to recognize a shape of the lesion area based on the lesion map, determine an output pattern of therapeutic light based on the recognized shape of the lesion area, and output patterned therapeutic light based on the determined output pattern,
wherein the shape of the lesion area corresponds to an output pattern of the patterned therapeutic light.

14. The light-based diagnostic treatment apparatus of claim 13, wherein the therapeutic light generator is configured to:
determine an intensity of the patterned therapeutic light to be output to each predetermined position in the lesion area based on an accumulated amount of the patterned therapeutic light at each predetermined position in the lesion area displayed on a cumulative light irradiation map, and
output therapeutic light having the determined intensity of therapeutic light through the optical stimulation unit.

15. A light-based diagnostic treatment apparatus, comprising:
a lesion detector configured to obtain a first lesion image of a lesion area of a user based on an optical signal transmitted through a single optical path, detect the lesion area from the obtained first lesion image, and in response to termination of detecting the lesion area, transmit a first trigger signal to a therapeutic light generator; and
the therapeutic light generator configured to in response to reception of the first trigger signal, generate patterned therapeutic light based on a shape of the lesion area, and output the patterned therapeutic light to the lesion area through the single optical path.

16. The light-based diagnostic treatment apparatus of claim 15, wherein the therapeutic light generator is configured to, in response to termination of outputting the patterned therapeutic light, transmit a second trigger signal to the lesion detector, and
the lesion detector is configured to, in response to reception of the second trigger signal, obtain a second lesion image of the lesion area based on an optical signal transmitted through the single optical path and detect the lesion area from the obtained second lesion image.

17. The light-based diagnostic treatment apparatus of claim 15, wherein the therapeutic light generator is configured to:
determine an output pattern of therapeutic light based on a shape of the lesion area, and
generate patterned therapeutic light based on the determined output pattern,
wherein the shape of the lesion area corresponds to an output pattern of the patterned therapeutic light.
